(19) United States
(12) Patent Application Publication
(10) Pub. No.: US 2022/0160460 A1
(43) Pub. Date: May 26, 2022

(54) SURGICAL GUIDE PLATE AND PREPARATION METHOD THEREFOR

(71) Applicant: ZUGA MEDICAL CHENGDU CO., LTD, Chengdu (CN)

(72) Inventors: YUE LI, CHENGDU (CN); CHAN Q WANG, CHAGRIN FALLS, OH (US)

(73) Assignee: ZUGA MEDICAL CHENGDU CO., LTD, Chengdu (CN)

(21) Appl. No.: 17/441,959

(22) PCT Filed: Apr. 7, 2020
(86) PCT No.: PCT/CN2020/079513

§ 371 Date: Sep. 22, 2021

(30) Foreign Application Priority Data
Mar. 22, 2019 (CN) 201910222634.4

(51) Int. Cl. A61C 1/08; A61B 6/03; A61B 6/14; A61B 6/00; A61C 9/00

(52) U.S. Cl. CPC A61C 1/082; A61B 6/032; A61C 9/0053; A61B 6/4085; A61C 9/0006; A61B 6/14

(57) ABSTRACT

Disclosed are a surgical guide plate and a preparation method therefor. The preparation method comprises the following steps: a) bonding a radiation-blocking material onto an oral mucosa, and putting on an occlusal rim; b) carrying out CBCT photographing to acquire CBCT data carrying information of the radiation-blocking material; c) acquiring STL data of an oral cavity surface; d) matching the CBCT data with the STL data; e) loading occlusal rim data; f) carrying out intelligent tooth arrangement; and g) designing an implant, and generating a surgical guide plate. By means of the preparation method, the level of comfort of a patient can be increased, preparatory processes are reduced, a radiation guide plate does not need to be made, the number of return visits is reduced, the clinical medical cost is reduced, the precision of a surgical guide plate is increased, the software design time is reduced, and the treatment experience is improved.

### TECHNICAL FIELD

This invention relates to a surgical guide and a preparation method thereof, which can be applied to oral implantation, orthodontics, surgery, repair and other operations requiring simultaneous integration of soft tissue data and convenient tooth arrangement. It is especially suitable for making surgical guides on implants, which belongs to the dental field.

### TECHNICAL BACKGROUND

In oral clinical patients, many old patients with missing teeth and poor bone condition are often encountered. At present time, making a tissue-supported surgical guide can greatly help dentists to accurately place an implant, while giving patients little trauma. The preparation of the traditional tissue-supported surgical guide is quite complicated, and the patient needs to make a scan appliance (similar to the process of making removable dentures) and visit the dentist several times before the dentist can get the complete information required, which will in turn increase the cost and radiation dosage of the patient. At the same time, dentists need to spend a lot of clinical time to prepare for the work, which also affects the process speed of the cases. In the traditional method, all data sources are based on Conical Beam Scanning CT (CBCT), but the accuracy of the current CBCT equipment is in the range of 0.2-0.5 mm, which cannot fully meet the requirements of high precision of surgical guide.

In order to improve the precision of implantation and implement minimally invasive surgery, more and more dentists in the dental field hope to utilize surgical guides for guided surgery. However, it is very complicated to use the traditional method to make a surgical guide when the patient is edentulous or missing a large number of teeth.

In existing technology, the dentist needs to take routine CBCT first, and records the relationship between the conventional impression and the jaw position of the patient, and then sends the information to the processing lab to make the scan appliance. After receiving the scan appliance, the dentist takes a second CBCT image of the patient, and at the same time, the scan appliance by itself separately. Then the data of the two CBCT images are transmitted into the software for the design of the surgical guide. The procedure is as follows:

1. taking the impression model of the patient;

2. obtaining the jaw position relationship of patients;

3. making scan appliance according to information 1-2;

4. taking CBCT image of the patient with scan appliance;

5. taking CBCT image of the scan appliance separately;

6. importing the data obtained in 4 and 5 into the design software to design the surgical guide;

7. printing the surgical guide.

The disadvantages of the method of preparing surgical guide with existing technology lie in that the preparation of traditional tissue-supported scan appliance is quite complicated, and the patient needs to visit many times before the dentist can get the complete required information, which will effectively increase the cost and radiation dosage of the patient. At the same time, dentists need to spend a lot of clinical time to prepare for the work, which also affects the process speed of the cases. All data sources are based on CBCT, and the current accuracy of CBCT equipment is mostly in the range of 0.2-0.5 mm, which cannot fully meet the requirements of high precision of a surgical guide. Therefore, there is an urgent need for a precise and convenient method to improve the efficiencies of such cases.

### Contents of the Invention

The present invention provides a surgical guide and its preparation method thereof, through the use of intelligent occlusal embankment implementation to get the patients oral CBCT data and STL data with one visit, can significantly improve the patient's comfort, reduce the preparatory process, eliminate the need to make a scan appliance, reduce the number of follow-ups, clinical medical treatment cost, improve the surgical guide accuracy, reduce the design time during software design and greatly improve the medical experience.

In order to solve the above technical problems, the invention adopts the following technical scheme:

(1) A method for preparing a surgical guide, including the following steps:

a) bonding a radiation-blocking material, and put on an intelligent occlusal rim; The ideal bite height and facial fullness can be restored by the insertion of occlusal embankments

b) carrying out CBCT photographing to acquire CBCT data;

c) acquiring STL data of an oral cavity surface;

d) matching the CBCT data with the STL data;

e) loading the occlusal rim data;

f) carrying out intelligent tooth arrangement;

g) designing implant placements and generating a surgical guide.

(2) According to the preparation method of the surgical guide described in (1), it also includes the bonding of a radio blocking material to a healthy tooth.

(3) According to the preparation method of the surgical guide as described in (1) or (2), at least four points on the oral mucosa are bonded with the radio blocking material.

(4) According to the preparation method of the surgical guide described in (1)-(3), the reflective material is bonded on the occlusal embankment. When the number of healthy teeth is small, for example, less than five, the reflective material needs to be bonded on the occlusal bank.

(5) According to the preparation method of surgical guide described in (1)-(4), a tissue support tray is placed on the oral mucosa prior to the insertion of the occlusal embankment. This ensures that the occlusal embankment can be fixed and reset accurately with a tissue-supported tray.

(6) According to the preparation method of surgical guide described in (1)-(5), the injection blocking material is bonded on the occlusal embankment. Mark the position of the canines on both sides with a linear blocking material.

(7) According to the preparation method of the surgical guide described in (1)-(6), the obstruction material is point-shaped, line-shaped or stripe-shaped.

(8) The height of the intelligent occlusal embankment is adjusted by adding pads on the occlusal embankment according to the preparation method of surgical guide described in any of the items (1)-(7), in order to achieve the ideal occlusal height of patients, the facial fullness, and temporomandibular surface and joint comfort.

(9) According to the preparation method of surgical guide described in (1)-(8), tissue support tray is prepared by any combination of one or at least two of the following materials: wax, plastic, PEEK, silicone rubber, thermal material, resin, and photosensitive resin.

(10) According to the preparation method of the surgical guide described in (1)-(9), the occlusal embankment is made of one or at least two of the following materials: wax, plastic, PEEK, silicone rubber, thermal material, resin, and photosensitive resin.

(11) According to the preparation method of the surgical guide described in (1)-(10), obtaining oral surface STL data method includes:

(1) When the oral impression is taken, the blocking material will leave an impression on the corresponding position of the impression material. After the impression is hardened, the blocking material is copied to the impression and the oral surface scanning equipment is used to scan the oral impression to obtain the STL data with the information of the blocking material;

Or 2) STL data with radiation blocking material information can be obtained by scanning directly in the mouth with oral surface scanning equipment;

Or 3) in oral mucosa at the most thick and thin adhesive resistance materials, and then according to CBCT, at this time of CBCT data shows that the bones and teeth and floating radio blocking materials information, due to the floating radio blocking materials on the soft tissue, by analyzing the relationship between radio blocking materials and bone, can obtain thickness, the relationship between bone and mucosa and soft tissue information, Finally, STL data is obtained.

(12) According to the preparation method of surgical guide described in either item (1)-(11), the step d) matching CBCT data with STL data: The CBCT data and STL data were integrated, and the information of the radiation blocking material was used as the matching basis. By adjusting the dental tissue information in the CBCT data and STL data, the jaw information, tooth tissue information and soft tissue information were finally matched.

(13) According to the preparation method of surgical guide as described in (1)-(12), step e) loading data of occlusal embankment: loading data of occlusal embankment according to the model of occlusal embankment.

(14) According to the preparation method of surgical guide mentioned in any item (1)-(13), the step f) intelligent tooth arrangement: select the appropriate tooth size for the patient, and automatically generate tooth arrangement information according to the data of occlusal embankment and tooth size.

(15) According to the preparation method of surgical guide mentioned in (1)-(14), step g) implant design and surgical guide generation: Implant placement location is designed according to tooth row information, and then the surgical guide is generated by combining soft tissue information and implant location information.

(16) an intelligent occlusal embankment, which comprises a freely shaped intelligent occlusal embankment and a gasket; The intelligent occlusal embankment is composed of multiple sections, each of which contains at least four fixed radiating points, and gaskets are detachably connected to the intelligent occlusal embankment. The shape data of each segment of the intelligent occlusal embankment, the location of radial points and the data of gaskets are preset in the software in advance.

(17) According to the intelligent occlusal embankment mentioned in (16), the intelligent occlusal embankment is provided with a screw hole, and the gasket is provided with a screw rod, through which the intelligent occlusal embankment and gasket are fixedly connected.

(18) According to the intelligent occlusal embankment mentioned in (16) or (17), the intelligent occlusal embankment is provided with a hole, and the gasket is provided with a bulge, and the intelligent occlusal embankment and gasket are fixedly connected through the hole and screw.

(19) According to the intelligent occlusal embankment mentioned in any item (16)-(18), the intelligent occlusal embankment and gasket are provided with adhesive materials, and the intelligent occlusal embankment and gasket are connected by adhesive materials.

(20) According to the intelligent occlusal embankment mentioned in any item (16)-(19), the intelligent occlusal embankment is prepared by any combination of one of the following materials or at least two materials: wax, plastic, PEEK, silicone rubber, thermal material, resin and photosensitive resin.

(21) The intelligent occlusal embankment according to any of items (16)-(20), wherein the intelligent occlusal embankment is in the shape of a bow, and the bow is a pointy round arc, oval arc or square arc.

(22) According to the intelligent clamping embankment mentioned in (16), the gasket shown is a gasket with screw or a gasket with hole, and the gasket with hole is sheathed on the gasket with screw.

(23) A surgical guide comprises a guide body and a guide ring, which includes an implant guide ring and an anchor pin guide ring.

(24) According to the surgical guide mentioned in (23), the main material of the guide is photosensitive resin, and the guide ring is stainless steel metal guide ring.

The invention provides a surgical guide and a preparation method thereof, and the beneficial technical effects are as follows:

(1) Due to the emergence of intelligent occlusal embankment, there is no need to make a scan appliance. This method can greatly improve the comfort of patients, reduce the preparation process, reduce the number of return visits, reduce clinical medical costs, and greatly improve the medical experience;

(2) Since there is no need to make a scan appliance, the oral surface scanning equipment is used to scan the oral surface data directly to improve the accuracy of the surgical guide;

(3) The special design of the software, combined with the intelligent occlusal embankment, can reduce the design time of tooth row and implant.

The definitions of relevant terms in the present invention are as follows:

1. CBCT refers to equipment such as conical beam scanning CT and 3D CT, which are used to obtain 3D image data information of oral cavity.

2. CBCT data refers to jaw, tooth tissue and other related information obtained by CBCT.

3. STL data refers to the information related to dental tissue and soft tissue in an oral cavity. High-precision oral surface scanning equipment can be used to perform fine scanning of oral tissue surface, and obtain STL data containing tooth tissue, soft tissue and other relevant information.

4. Scan appliance: a resin auxiliary device similar to removable denture, which can simultaneously develop tooth arrangement information in CBCT.

5. Surgical guide: it is used in oral implant surgery and fixed on the tooth or soft tissue. Implant drill and implant are inserted accurately according to the position of the guide.

### DESCRIPTION OF PICTURES

The following is a further detailed description of the invention in combination with the attached drawings and specific implementation methods.

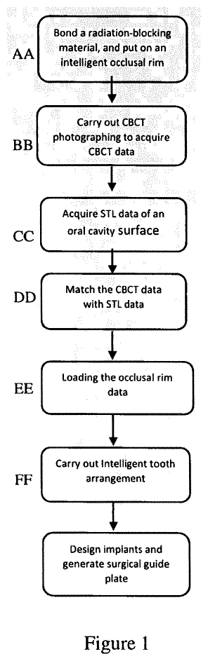

FIG. 1 is the flow chart of the preparation method of the surgical guide of the invention.

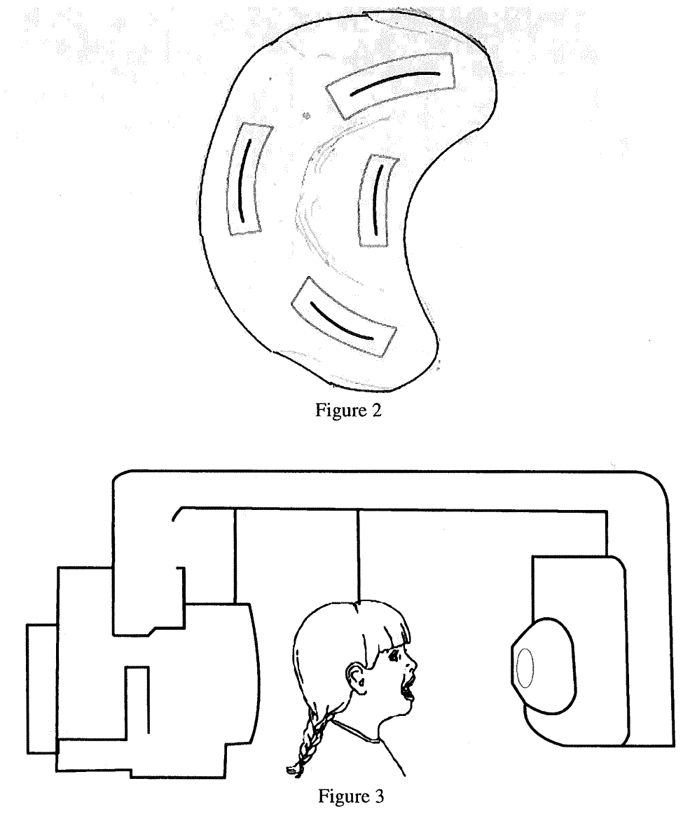

FIG. 2 is a schematic diagram of the adhesive material on the mucosal surface.

FIG. 3 shows the schematic diagram of CBCT after the patient was put in the occlusal embankment.

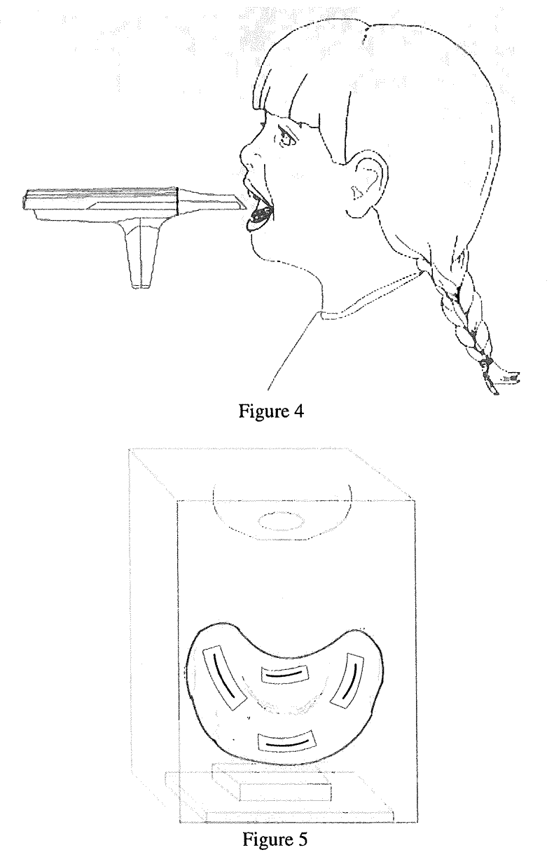

FIG. 4 is a schematic diagram of direct oral scanning with oral surface scanning equipment.

FIG. 5 is the schematic diagram of scanning oral impression with oral surface scanning equipment.

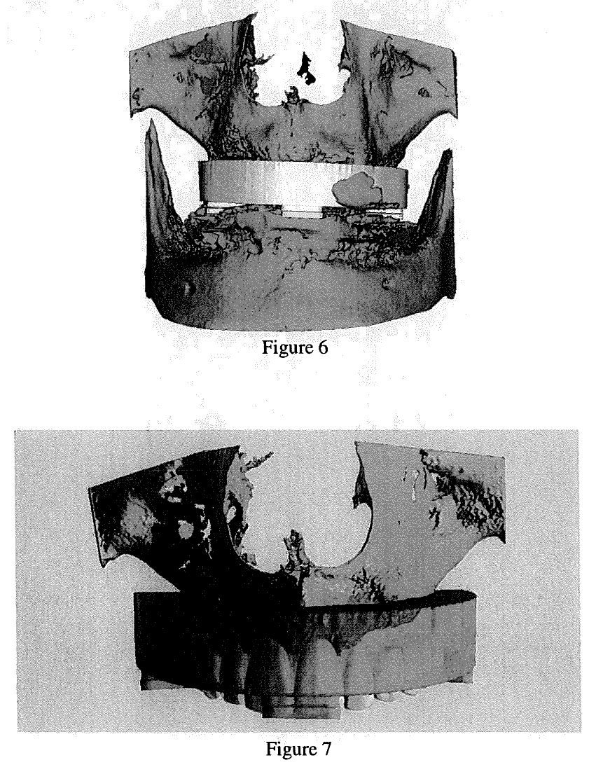

FIG. 6 shows the schematic diagram of loading the data of intelligent occlusal embankment.

FIG. 7 is a schematic diagram of intelligent tooth arrangement based on the data of intelligent occlusal embankment.

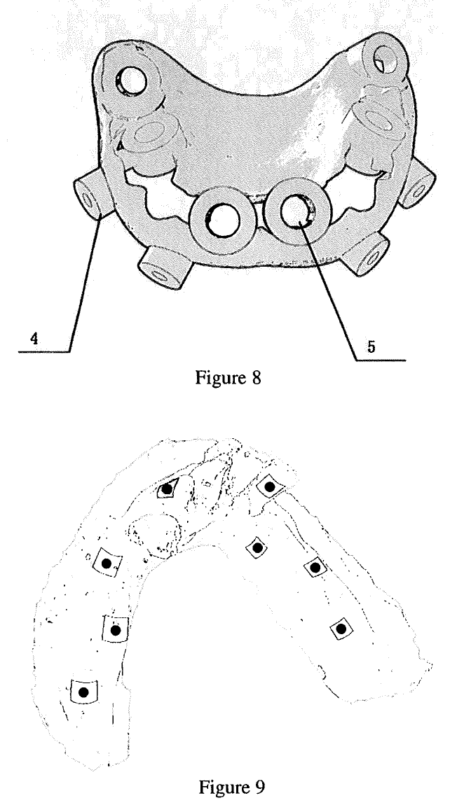

FIG. 8 is the schematic diagram of the invented surgical guide.

FIG. 9 is a schematic diagram of bonding the radio blocking material on the tooth.

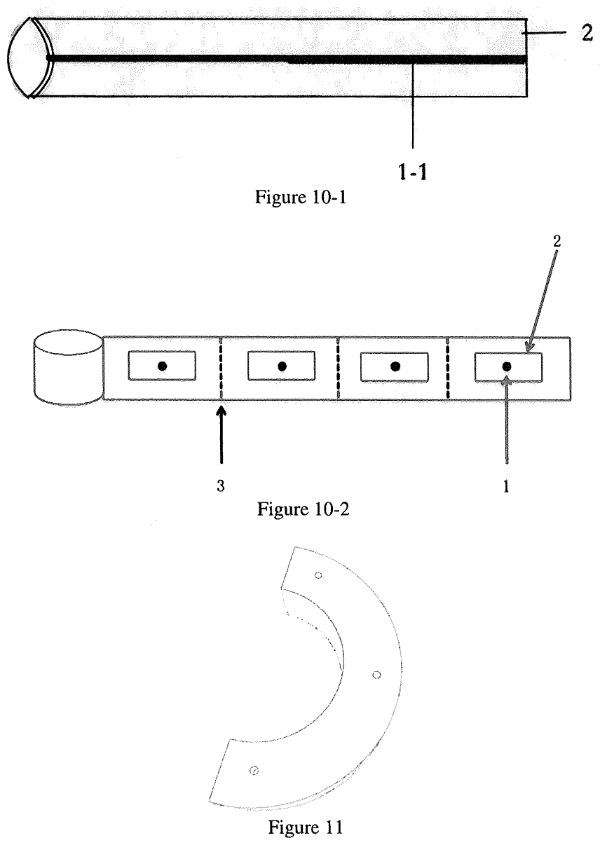

FIG. 10-1 is a schematic diagram of linear radio blocking materials.

FIG. 10-2 is a schematic diagram of point-like radio blocking materials.

FIG. 11 is the structural diagram of intelligent occlusal embankment.

FIGS. 12-1 and 12-2 are schematic diagrams of intelligent occlusal embankment with gaskets added.

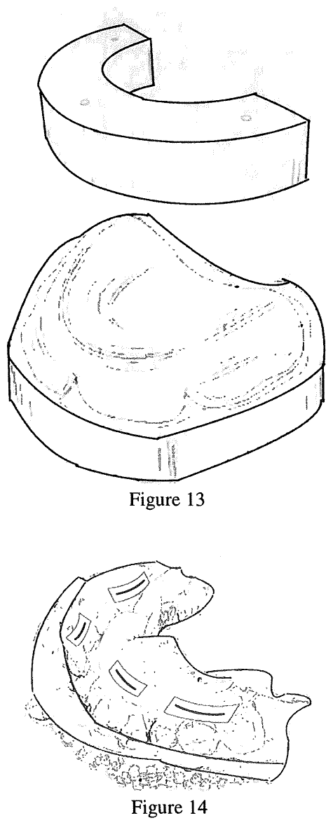

FIG. 13 Schematic diagram of the coordination between the tissue support tray and the intelligent occlusal embankment.

FIG. 14 shows the impression of the radio blocking material.

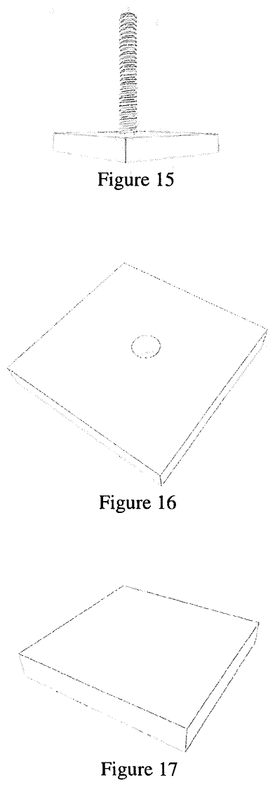

FIG. 15 is gasket with screw.

FIG. 16 shows gasket with hole.

FIG. 17 shows gasket with adhesive material.

### SPECIFIC IMPLEMENTATION EMBODIMENTS

### Embodiment 1

As shown in FIG. 1-9, a preparation method of surgical guide is as follows:

a) At least four points of the oral mucosa were glued with the projectile blocking material (as shown in FIG. 2), and the intelligent occlusal embankment was inserted; The ideal occlusal height and facial fullness could be restored by the insertion of occlusal embankment. With special materials: all kinds of plastic materials, such as wax, all kinds of plastic, PEEK, silicon rubber, thermal material, resin, photosensitive resin such as but not limited to one, two or more of these materials alone or with splint made of embankment, the intelligent occlusal embankment was inserted, the process may use wax knife, carving knife, screw wire cutter, etc. If the middle bite height is insufficient, spacers may be inserted on either side or in the middle until the client has recovered the desired bite height, facial fullness, and temporal-dental surface and joint comfort.

b) CBCT photography (as shown in FIG. 3) was conducted to obtain CBCT data with information of obstructed materials.

c) Obtaining oral surface STL data: Methods for obtaining oral surface STL data include: 1) taking the oral impression, and the blocking material will leave an imprint on the corresponding position of the impression material. After the impression is hardened, the blocking material is copied to the impression, and the oral surface scanning equipment is used to scan the oral impression (as shown in FIG. 5), and the STL data with the information of the blocking material can be obtained (as shown in FIG. 14). Or 2) scanning directly in the mouth with oral surface scanning equipment to obtain the STL data with the information of the radiation blocking material (as shown in FIG. 4); Or 3) in oral mucosa at the most thick and thin place affix radio blocking materials, and then take a CBCT image, at this time of CBCT data shows that the bones and teeth and floating radio blocking materials information, due to the floating radio blocking materials on the soft tissue, through analyze radio blocking materials and the relationship between bone, can analyze and thickness, the relationship between bone and mucosa and soft tissue, Finally, STL data is obtained.

d) Matching CBCT data with STL data: CBCT data and STL data are integrated, and the information of occlusal materials is used as the matching basis. Finally, jaw information, tooth tissue information and soft tissue information are matched by adjusting the tooth tissue information in CBCT data and STL data.

e) Loading data of occluded embankment (as shown in FIG. 6): Loading data of occluded embankment according to the model of occluded embankment.

f) Intelligent tooth arrangement (as shown in FIG. 7): Selecting the appropriate tooth size for patients, and automatically generate tooth arrangement information according to the data of occlusal embankment and tooth size.

g) Implant design and surgical guide generation: Implant placement position is designed according to tooth row information, and then surgical guide is generated by combining soft tissue information and implant location information (as shown in FIG. 8).

Preferably, a tissue-supported tray is placed on the oral mucosa prior to insertion into the occlusal embankment. As shown in FIG. 13, the tissue support tray can be a variety of plastic materials, such as wax, various plastics, PEEK, silicone rubber, thermal materials, resin, photosensitive resin, but not limited to one of these materials. As long as the soft tissue is fully attached, the embankment can be supported and reduced. After the occlusal embankment is inserted, the position of each cusp is marked with a linear radio blocking material.

Preferably, the reflective material is bonded to the healthy tooth (FIG. 9).

Preferably, when the number of remaining healthy teeth is small, e.g., less than five, the reflective material needs to be bonded on the occlusal bank.

Preferably, the blocking material is point-shaped, line-shaped or striated. As shown in FIG. 10-1, the linear radio blocking material 1-1 is set on the marking tape 2 during packaging. As shown in FIG. 10-2, the point radio blocking material 1 is arranged on the marking tape 2, and a cutting line 3 is arranged on the marking tape 2. According to different packaging requirements and production strategies, the radio blocking material can be packaged in roll, single sheet or multiple pieces.

### Embodiment 2

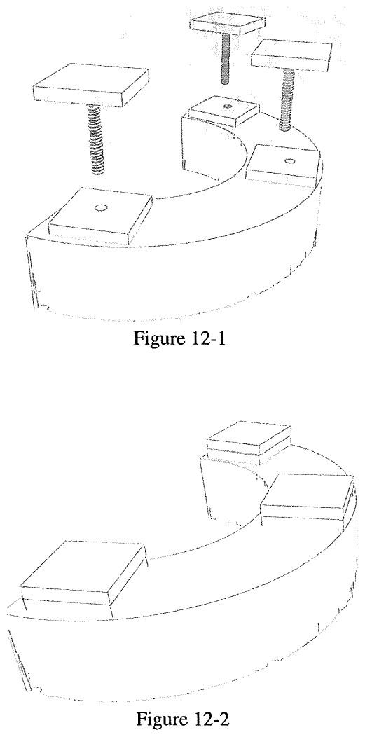

As shown in FIG. 11, 12-1, 12-2, 15-17, a kind of intelligent occlusal embankment consists of a free molding intelligent occlusal embankment (as shown in FIG. 11) and gaskets (as shown in FIG. 15-17). The gasket is detachably attached to the intelligent occlusal bank. As shown in FIG. 11, a screw hole is arranged on the intelligent occlusal embankment, and a screw is arranged on the gasket (as shown in FIGS. 12-1 and 15). The intelligent occlusal embankment and gasket are fixedly connected through the screw hole and screw. As shown in FIG. 12-2, bonding materials are provided on the intelligent occlusal embankment and gasket, and the intelligent occlusal embankment and gasket are connected by bonding materials. The intelligent occlusal embankment is prepared by any combination of one of the following materials or at least two materials: wax, plastic, PEEK, silicone rubber, thermal material, resin, photosensitive resin. Preferably, gaskets with holes are provided (as shown in FIGS. 12-1 and 16), and the gaskets with holes can be set on the gaskets with screws (as shown in FIG. 12-1), so as to adjust the height of the intelligent occlusal embankment.

The intelligent occlusal embankment is a bow, and the bow is a pointy round bow, an oval bow or a square shaped bow. This is designed according to the size of the patient's occlusal bone. At the same time, the height of intelligent occlusal embankment can be adjusted by adding gaskets.

A free plastic intelligent occlusal embankment with a maximum size of 10 mm*80 mm*100 mm, intelligent occlusal embankment is provided with sunken screw holes to facilitate the insertion of screw fixed gaskets. The gasket is divided into two kinds: gasket with screw and gasket with adhesive. Gaskets with set screws, each 5 mm thick, can be fixed to the occlusal embankment by screws. The spacers of fixed position are bonded, each spacer is 0.5 mm thick, and are bonded to the occlusal embankment by the bonding material. The gasket and the intelligent occlusal embankment materials contain the marking points of the blocking material. When the patient wears the occlusal embankment, the radiation points and the patient's jaw and other information develop simultaneously, but the intelligent occlusal embankment does not develop.

The intelligent occlusal embankment and gasket can be any color, or packaged individually, or packaged in multiple packages, etc., depending on packaging requirements and production strategies.

In addition, the implant software has a database of intelligent occlusal embankment in advance, and the shape and position of occlusal embankment can be reproduced in the software after the shape, model and gasket height of intelligent occlusal embankment are input. At the same time, the tooth library is placed in the software. When the intelligent tooth arrangement is clicked, the software can automatically calculate the tooth alignment according to the shape and position of the occlusal embankment.

### Embodiment 3

As shown in FIG. 8, a surgical guide consists of a guide body and a guide ring, which includes an implant guide ring 4 and a retainer needle guide ring 5. The guide body material is photosensitive resin, the guide ring is a stainless steel metal guide ring. The guide has smooth appearance, uniform color, no scars, no scratches, and smooth edge round blunt. The guide size minimum is 30*20 mm, the maximum 100*100 mm. The guide thickness is greater than or equal to 2.5 mm.

The above embodiments are not exhaustive examples of specific embodiments, there may be other embodiments, the purpose of the above embodiments is to explain the invention, but not to limit the scope of protection of the invention, all simple variations of the application of the invention fall within the scope of protection of the invention.

This patent specification uses examples to demonstrate the invention, including the best mode, and enables the invention to be manufactured and used by technicians familiar with the field. The scope of authorization of the invention includes the contents of the claim, the specific mode of implementation in the specification and the contents of other embodiments. Such other instances shall also fall within the scope of the patent claims for the invention provided that they contain technical features described in the same written language as the claims, or that they contain technical features described in similar literal language not materially different from the claims.

The entire contents of all patents, patent applications and other references shall be incorporated by reference into this application. However, if a term in this application conflicts with a term included in the bibliography, the term in this application takes precedence.

It is important to note that “first”, “second”, or similar terms do not indicate any order, quality, or importance, but are used to distinguish between different technical features. The modifier “approximately” used in conjunction with quantity contains the meaning indicated by the value and context of the content (e.g., it contains the error in measuring a particular quantity).

1. A method for preparing a surgical guide, the method comprising:
a) bonding a radiation blocking material on the oral mucosa and putting the radiation blocking material in an occlusal embankment;
b) taking CBCT photography to obtain information with the blocking material CBCT data;
c) obtaining the STL data of the oral cavity surface;
d) matching the CBCT data with the STL data;
e) loading occlusal embankment data;
f) arranging tooth intelligently; and
g) designing implant placements and generating a surgical guide.

2. The method for preparing a surgical guide according to claim 1, the method further comprising:
adhering the radiation blocking material to a healthy tooth.

3. The method for preparing a surgical guide according to claim 1, wherein at least four points on the oral mucosa are bonded with the radiation blocking material.

4. The method for preparing a surgical guide according to claim 1, wherein the radiation blocking material is bonded to the occlusal embankment.

5. The method for preparing a surgical guide according to claim 1, wherein a tissue support tray is placed on the oral mucosa before putting on the occlusal embankment.

6. The method for preparing a surgical guide according to claim 5, wherein the radiation blocking material is adhered to the tissue support tray.

7. The method for preparing a surgical guide according to claim 1, wherein the radiation blocking material is in the shape of dots, lines or stripes.

8. The method for preparing a surgical guide according to claim 1, wherein a height of the occlusal embankment is adjusted by adding a spacer on the occlusal bank embankment.

9. The method for preparing a surgical guide according to claim 5, wherein the tissue support tray is made of one of the following materials or any combination of at least two materials: wax, plastic, PEEK, silicone rubber, heat sensitive material, resin, and photosensitive resin.

10. The method for preparing a surgical guide according to claim 1, wherein, in c), the obtaining comprises:
1) taking an oral impression, and the radiation blocking material will leave a mark on the corresponding position of the impression material, wherein, after the impression is hardened, copying the radiation blocking material onto the impression, and scanning the oral impression with an oral surface scanning device to obtain STL data with information on radiation blocking materials;
or 2) using the oral surface scanning device to scan directly in the oral cavity to obtain STL data with the information of the radiation blocking material;
or 3) bonding the radiation blocking material to the thickest and thinnest part of the oral mucosa, and taking the CBCT, at this time the CBCT data shows bone, tooth information and suspended radiation blocking material information;
wherein, since the suspended radiation blocking material is attached to the soft tissue, by analyzing the relationship between the radiation blocking material and bone, the relationship and thickness of bone and mucosa is analyzed, to obtain soft tissue information, and finally obtain STL data.

11. The method for preparing a surgical guide according to claim 1, wherein:
in d), the matching includes integrating the CBCT data and STL data with radiation blocking material information being used as the basis for matching, wherein, by adjusting the tooth tissue information in the CBCT data and STL data, the jaw information, tooth tissue information, and soft tissue information are finally matched;
in e), the loading includes loading the occlusal embankment data according to the model of the occlusal embankment;
in f) the arranging includes selecting the tooth size suitable for the patient, and automatically generating tooth arrangement information according to the occlusal embankment data and the tooth size data;
in g), the designing includes designing the placement position of the implant according to the tooth arrangement information; and
combining the soft tissue information and the implant position information to generate the surgical guide.

12. An intelligent occlusal embankment, comprising:
a freely shaped occlusal embankment and
a gasket;
wherein the gasket is detachably connected to the freely shaped occlusal embankment.

13. The intelligent occlusal embankment according to claim 12, wherein the freely shaped occlusal embankment is provided with a screw hole, the gasket is provided with a screw, and the freely shaped occlusal embankment is connected with the gasket through the screw hole and the screw, making the freely shaped occlusal embankment and the gasket a fixed connection.

14. The intelligent occlusal embankment according to claim 12, wherein the freely shaped occlusal embankment is provided with a hole, the gasket is provided with a hole, and the freely shaped occlusal embankment and the gasket are formed through the hole and a screw with a fixed connection.

15. The intelligent occlusal embankment according to claim 12, wherein the freely shaped occlusal embankment and the gasket are both provided with adhesive materials, and the freely shaped occlusal embankment and the gasket are connected by the adhesive material.

16. The intelligent occlusal embankment according to claim 12, wherein the gasket is provided with a screw or a second gasket is provided with a hole, and the second gasket with the hole is sleeved on the gasket with the screw.

17. The intelligent occlusal embankment according to claim 12, wherein the freely shaped occlusal embankment is made of one of the following materials or any combination of at least two materials: wax, plastic, PEEK, silicone rubber, thermal sensitive materials, resins, photosensitive resins.

18. The intelligent occlusal embankment according to claim 12, wherein the intelligent occlusal embankment is arcuate, and the arcuate shape is a pointed round arc, oval arc, or square circular arc.

19. A surgical guide, comprising:
a guide main body; and
a guide ring;
wherein the guide ring includes an implant guide ring and an anchor pin guide ring.

20. The surgical guide according to claim 19, wherein the main body of the guide is photosensitive resin, and the guide ring is a stainless steel metal guide ring.
